(19) United States
(12) Patent Application Publication
(10) Pub. No.: US 2010/0331637 A1
(43) Pub. Date: Dec. 30, 2010

(54) Method and System for Measuring a Composition in a Blood Fluid

(75) Inventors: Choon Meng TING, Singapore (SG); Paramesran RAVEENDRAN, Malaysia (MY)

Correspondence Address: INTELLECTUAL PROPERTY LAW GROUP LLP, 12 SOUTH FIRST STREET, SUITE 1205, SAN JOSE, CA, 95113, US

(73) Assignee: GLUCOSTATS SYSTEM PTE. LTD., Singapore (SG)

(21) Appl. No.: 12/918,315

(22) PCT Filed: Apr. 13, 2009
(86) PCT No.: PCT/SG2009/000135

§ 371 Date: Aug. 18, 2010

(30) Foreign Application Priority Data
Apr. 16, 2008 (SG) 200802911-8

(51) Int. Cl. A61B 5/1455

(52) U.S. Cl. 600/316

(57) ABSTRACT

A system (10) and method for measuring a composition in the blood fluid is disclosed. The system (10) comprises a non-invasive measuring unit (12) for measuring the composition; and at least one neural network (16) for processing a plurality of measurements taken by the non-invasive measuring unit (12) to determine an overall measurement of the composition in the blood fluid. A further aspect of the invention discloses a computer-readable medium for performing the above method.

### FIELD OF THE INVENTION

The present invention relates to a method and system for measuring a composition in the blood fluid. The invention is particularly suited to processing a set of blood glucose measurements of a person through at least one neural network to obtain an overall blood glucose level and will be described in this context.

### BACKGROUND TO THE INVENTION

The following discussion of the background of the invention is intended to facilitate an understanding of the present invention. However, it should be appreciated that the discussion is not an acknowledgement or admission that any of the material referred to was published, known or part of the common general knowledge in any jurisdiction as at the priority date of the application.

A traditional way of measuring a person's blood glucose level is to use a fine needle to prick the finger of a person. This invasive technique then allows blood from the person's veins to be drawn through the incision caused by the needle. This blood is then placed on a strip containing reagents that react with glucose to form a chromophore. The strip is subsequently read by a reflectance colorimeter with an analyser (e.g. a glucose meter) to determine the level of glucose present in the blood.

Such invasive approaches are undesirable in situations where the person is required to monitor his/her blood glucose level several times a day. This is due to the fact that taking multiple measurements in this manner:
can cause unnecessary pain and hassle;
increases the risk of contamination in situations where needles are re-used. Conversely, if needles are not reused, the cost of needle disposal is increased in line with the number of measurements required to be taken on a daily basis; AND
Bio-waste products are increased in line with the number of measurements required to be taken on a daily basis which must then be appropriately dealt with.

These problems with invasive techniques have led to the development of non-invasive techniques for measuring blood glucose levels.

Out of the various non-invasive monitoring techniques, the optical absorption technique for the quantification of glucose has demonstrated to be a promising approach for non invasive blood glucose sensing/monitoring. The optical absorption technique principle centres on the use of an incident infrared radiation source of a certain wavelength being delivered to a measurement site through optical fibres. The wavelength of the infrared radiation is such that it is prone to absorption by glucose in the blood fluid.

Thus, as the infrared radiation is directed through the measurement site, part of the radiation will be absorbed or reflected by glucose in the blood fluid to an optic fibre sensor. The amount of infrared radiation measured by the sensor is then used to compute a glucose level. To eliminate error in this process, additional optic fibre sensors may surround the sensor and communicate the level of infrared radiation each receives to the main sensor for inclusion in its computations.

The problems introduced by non-invasive blood glucose measurement systems are many. In the case of the optical absorption technique described above, the problems include:
differences in the pressure applied by the optical fibres affect the blood glucose measurement obtained. Accordingly, it is possible to obtain differing blood glucose level measurements from the same measurement site at different times. It is also possible for variations in sequential blood glucose measurements to arise as a result of variations in pressure between the two measurements;
The wavelengths used can be prone to soft tissue interference, which may result in higher blood glucose measurements;
The skin type of the person may affect the ability of the infrared radiation to penetrate tissue or may absorb the infrared radiation, again adversely affecting the accuracy of the resulting blood glucose measurement.
The wavelength chosen may be prone to absorption by other elements in the blood fluid, such as urea, water, etc. in addition to blood glucose.

One method of dealing with the immediately preceding problems has been to implement systems relying on a plurality of infrared radiation beams of different wavelengths to measure the blood glucose level. These measurements are then processed using a partial least-square method for calculating the blood glucose level.

The problem with this situation, however, is that the accuracy of the blood glucose measurement is reliant on the number of differing wavelengths used to take the measurement. While greater numbers of differing wavelengths improve such accuracy, they do so at increased cost. The end result has seen a situation where accuracy corresponding to that of invasive techniques has not been able to be obtained through non-invasive measurement techniques based on the optical absorption principle at a cost effective level.

It is thus an object of the present invention to develop a system capable of measuring and determining blood fluid composition such as glucose, while ameliorating the above-mentioned problems thereby attempting to achieve a balance between cost and accuracy.

### SUMMARY OF THE INVENTION

Throughout this document, unless otherwise indicated to the contrary, the phrase “comprising”, “consisting of”, and the like, are to be construed as inclusive and not exhaustive.

In accordance with a first aspect of the invention there is a system for measuring a composition of a blood fluid comprising at least one neural-network for processing a plurality of measurements taken by a non-invasive measuring unit to determine an overall measurement of the composition in the blood fluid.

In accordance with a further aspect of the invention there is a system for measuring a composition of a blood fluid comprising a non-invasive measuring unit for measuring the composition; and at least one neural network for processing a plurality of measurements taken by the non-invasive measuring unit to determine an overall measurement of the composition in the blood fluid.

In accordance with a further aspect of the invention there is a method of measuring a composition in a blood fluid comprising the steps of obtaining a plurality of measurements from a non-invasive measuring unit and processing the plurality of measurements by at least one neural network to determine an overall measurement of the composition in the blood fluid.

Preferably, the at least one neural network implements a back propagation algorithm.

The number of nodes in the input layer preferably matches the number of measurements in the plurality of measurements taken by the non-invasive measuring unit. Further, preferably the hidden layer comprises at least four nodes.

A linear equation associated with each output node may be determined from a controlled source prior to training of the at least one neural network. The linear equation associated with each hidden node may be determined through automated processes.

The output value for the hidden node can be a summation of weighted measurements. The output value for the output node also can be a summation of weighted normalized hidden node output values.

The adjustment to the weightings for each link between a hidden node and an output node may be calculated with reference to an output gradient error. The output gradient error can be calculated as follows:

δₖ=(tₖ−nₖ)·nₖ·(1−nₖ)
where:
   nₖ is the normalized output value for output node k.
   tₖ is the target output value for output node k as determined by the linear equation associated with output node k.

The adjustment to the weightings for each link between a hidden node and an output node are calculated according to the formula:

Δwhoⱼₖ(p+1)=η·δₖ·f(netⱼ)+mΔwhoⱼₖ(p)
where:
   η denotes the learning rate.
   m denotes the momentum composition.
   δₖis the output gradient error.
   Δwhoⱼₖ(p+1) represents the updated change in weight.
   Δwhoⱼₖ(p) represents the previous change in weight.
   f(netⱼ) is the normalized output value for hidden node j.

The adjustment to the weightings for each link between an input node and a hidden node are preferably calculated with reference to a hidden layer gradient error. The hidden layer gradient error is calculated as follows:

$${δ}_{j} = ( f ( {net}_{j} ) \cdot ( 1 - f ( {net}_{j} ) ) ) {\sum }_{k = 1}^{Y} {δ}_{k} \cdot {who}_{jk} ( p )$$

where:
Y is the total number of neurons in the output layer of the neural network concerned.
   f(netⱼ) is the normalized output value for hidden node j.
   δₖ is the output gradient error.
   whoⱼₖ(p) represents the current weight for the link between the hidden node j and the output node k.

The adjustment to the weightings for each link between an input node and a hidden node may be calculated as follows:

Δwih_{if}(p+1)=η·δⱼ·xᵢ+mΔwihᵢⱼ(p)
where:
   η denotes the learning rate.
   m denotes the momentum composition.
   δⱼ is the hidden layer gradient error.
   xᵢ is the value of input node i.
   wihᵢⱼ(p+1) represents the updated change in weight.
   wihᵢⱼ(p) represents the previous change in weight.

The learning rate (η) and the momentum parameter (m) may be automatically adjusted during training. Preferably, the learning rate (η) is a value in the range 0.01 to 0.1 and the momentum parameter (m) is a value in the range 0.8 to 0.9.

Ideally, the at least one neural network comprises at least one bias.

The output value for the hidden node may be a summation of weighted measurements and at least one weighted input bias.

The output value for the output node may also be a summation of weighted normalized hidden node output values and at least one weighted output bias.

The adjustments to the weightings of each link between each output bias and an output node may be calculated with reference to the output gradient error. Ideally, this is through use of the following equation:

Δwho₀ₖ=η·δₖ
where:
   η is the learning rate.
   δₖis the output gradient error.

The adjustment to be made to the output value for the output node (netoₖ) can be determined by the following equation:

$${neto}_{k} = {who}_{{0}^{k}} \cdot {bo}_{k} + {\sum }_{j = 1}^{X} {who}_{jk} \cdot f ( {net}_{j} )$$

where:
   X is the total number of nodes in the hidden layer of the neural network concerned
   who₀ₖ is the weighting applied to the output bias for output node k.
   boₖ is the output bias for output node k
   whoⱼₖ is the weighting applied to the link between hidden node j and output node k.
   f(netⱼ) is the normalized output value for hidden node j.

Ideally, the at least one neural network comprises a first neural network and a second neural network, the first neural network configured so as to pre-process the plurality of measurements before passing the pre-processed measurements to the second neural network for determination of an overall measurement of the composition. The first and second neural networks may both implement back propagation algorithms. The back propagation algorithm implemented by the first neural network may be the same as that implemented by the second neural network.

The at least one neural network may be trained until one of the following occurs: the mean square error per training set is within a predetermined range; the synaptic weights stabilise; the bias level stabilises; the mean square error of the system is within a predetermined range; the mean square error over the entire training set is within a predetermined range; a predetermined number of training iterations have been performed. In a preferred embodiment, the at least one neural network is trained until the global mean square error of the system is less than 0.0008.

After training of the at least one neural network, the neural network(s) may be verified by comparing the results of the trained neural network against measurements of the substance obtained through invasive measuring techniques.

The non-invasive measuring unit may comprise a plurality of laser diodes each emitting light at a unique wavelength absorbable by the composition, the measurements taken by each laser diode forming the plurality of measurements.

The composition to be measured is preferably blood glucose and the wavelength of the light emitted by each of the plurality of laser diodes falls within the range 1600 nm to 1800 nm.

Alternatively, the non-invasive measuring unit comprises at least one laser diode able to emit light at varying wavelengths absorbable by the composition, the measurements taken by the at least one laser diode at each of these varying wavelengths forming the plurality of measurements.

The non-invasive measuring unit may further include a control laser diode which emits light at a wavelength not absorbable by the composition.

In accordance with a further aspect of the invention, there is a computer-readable medium having recorded thereon a means for receiving a plurality of measurements of a composition of a blood fluid, and at least one neural network to process the plurality of measurements of the composition of the blood fluid, such that an overall measurement of the composition in the blood fluid is determined.

### BRIEF DESCRIPTION OF THE DRAWINGS

The following invention will be described with reference to the following drawings of which:

FIG. 1 is a schematic representation of a system for measuring a composition in the blood fluid

FIG. 2 is a schematic of a first neural network forming part of the system shown in FIG. 1.

FIG. 3 is a series of glucose concentration graphs from which linear equations are manually determined for the purposes of training the first neural network as shown in FIG. 2.

FIG. 4 is a schematic of a second neural network forming part of the system shown in FIG. 1.

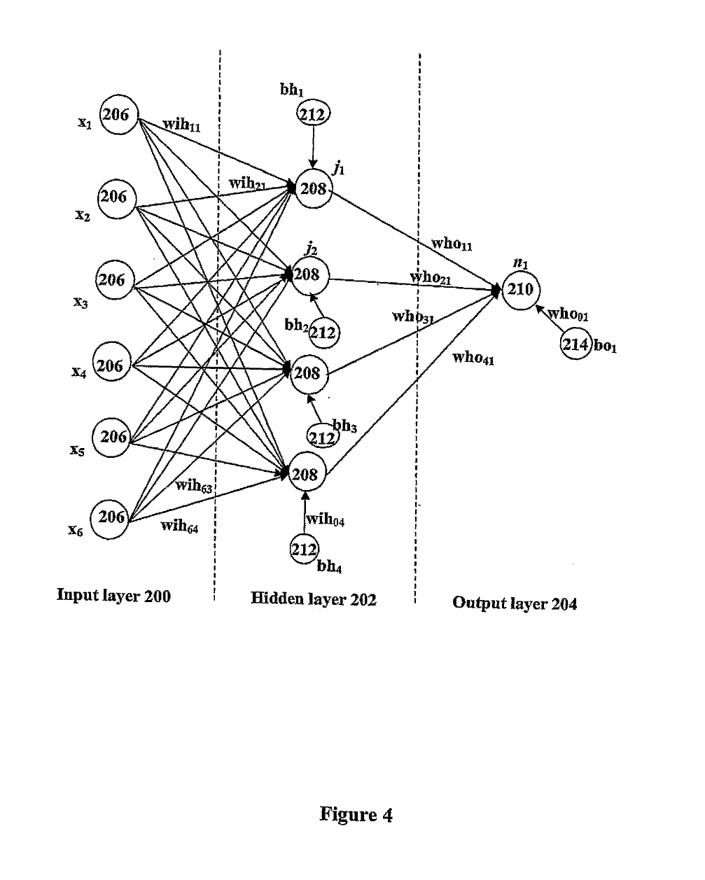

FIG. 5 is an isometric view of one version of a non-invasive blood glucose measurement setup.

### DETAILED DESCRIPTION OF THE INVENTION

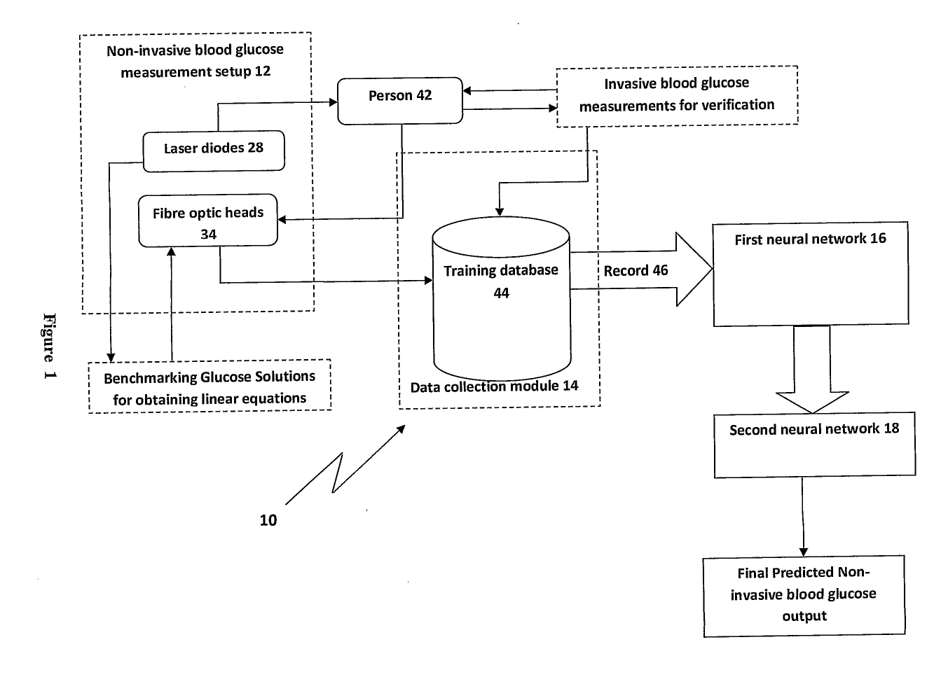

FIG. 1 illustrates the first embodiment of the system 10 for measuring blood glucose in the blood fluid 42. The system 10 comprises a non-invasive blood glucose measurement setup 12, a data collection module 14, a first neural network 16 and a second neural network 18. In the context of the invention blood fluid is composed of blood cells suspended in a liquid called blood plasma. Plasma, which comprises 55% of blood fluid, is mostly water (about 90%), and contains dissolved proteins, glucose, mineral ions, hormones, carbon dioxide, platelets and blood cells themselves. The blood cells present in blood are mainly red blood cells (also called RBCs or erythrocytes) and white blood cells, including leukocytes and platelets (also called thrombocytes). Blood fluid is the main medium for excretory product transportation within vertebrates in vivo. The blood fluid may be measured in situ through a nail or it may be extracted and measured in a capillary in vitro.

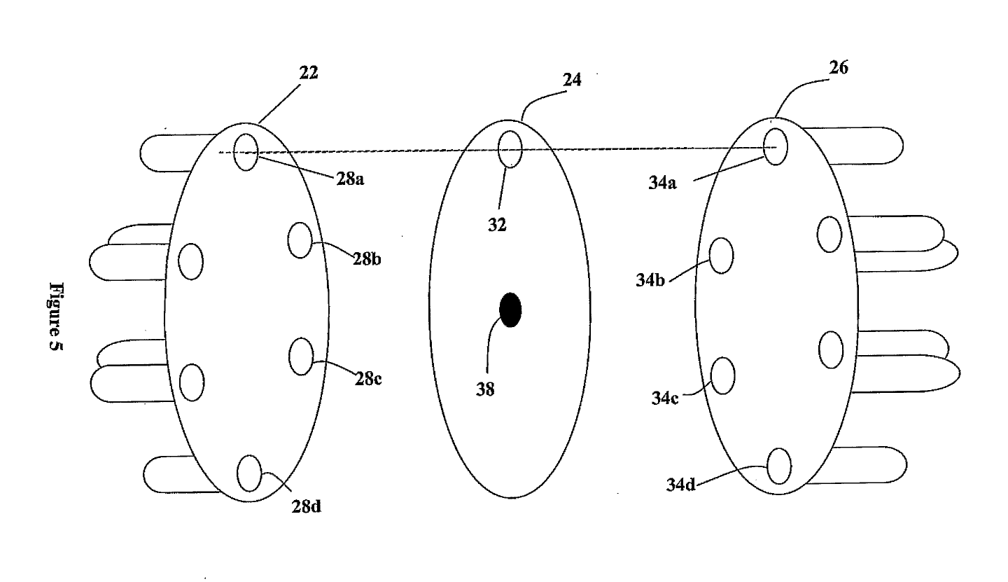

The non-invasive blood glucose measurement setup 12 comprises a source disc 22, a selector disc 24 and a detector disc 26. The selector disc 24 is positioned between the source disc 22 and the detector disc 26. The non-invasive blood glucose measurement setup 12 is shown in FIG. 5.

Source disc 22 has six laser diodes 28 attached thereto. The six laser diodes 28 are uniformly spaced about the circumference of the source disc 22. Each laser diode 28 is oriented in the same direction as each other laser diode 28.

Each laser diode 28 is configured to emit a single infrared wavelength in the range of 1600 nm to 1800 nm. No laser diode 28 emits an infrared wavelength identical to that of any other laser diode 28.

Selector disc 24 is rotatable about axle 38. Selector disc 24 has an aperture 32 offset from axle 38. In this manner, when rotated, the aperture 32 in the selector disc 24 allows the infrared beam emitted by each of the laser diodes 28 to pass therethrough. The aperture 32 is sized such that only one infrared beam emitted by a laser diode 28 can pass therethrough at any one time. A securing means (not shown in the figure) maintains the position of the selector disc 24. The securing means in this embodiment takes the form of a releasable clip. Thus when the clip engages the selector disc 24, the selector disc 24 can not rotate, but when the clip is released from the selector disc 24, the selector disc 24 is free to rotate about axle 38.

The detector disc 26 has six fibre optic heads 34 mounted thereon. The fibre optic heads 34 are arranged in an identical fashion to the laser diodes 28. This allows for axial alignment between each fibre optic head 34 with its corresponding laser diode 28 To elaborate, fibre optic head 34a is axially aligned to laser diode 28a, fibre optic head 34b is axially aligned to laser diode 28b, and so on.

Each fibre optic head 34 is in data communication with the data collection module 14. The data collection module 14 is in turn in data communication with the first neural network 16. The first neural network 16 is in turn in uni-directional data communication with the second neural network 18. In this example, the first neural network 16 comprises an input layer 100, a hidden layer 102, and an output layer 104. The input layer 100 consists of six input neurons 106. Each input neuron 106 is in communication with each hidden neuron 108 in the hidden layer 102. Each hidden neuron 108 is in turn connected to each output neuron 110 in the output layer 104. In addition, there is a bias input 112 in the input layer 100 and bias input 114 in the hidden layer 102. The values for the bias inputs 112, 114 are initially set at +1.

The second neural network 18 comprises an input layer 200, a hidden layer 202, and an output layer 204. The input layer 200 consists of six input neurons 206. Each input neuron 206 is in communication with each hidden neuron 208 in the hidden layer 202. Each hidden neuron 208 is in turn connected to the sole output neuron 210 in the output layer 204. In addition, there is a bias input 212 in the input layer 200 and bias input 214 in the hidden layer 202. The values for the bias inputs 212, 214 are initially set at +1.

The connections between each input neuron 206 and each hidden neuron 208 is weighted. As shown in the accompanying figures and equations, this weighting is designated wihᵢⱼ with i representative of the input neuron 206 connected and j representative of the hidden neuron 208 connected.

The invention will now be described in the context of its operation. Additional features necessary to the operation of the system 10 may also be introduced in the context of the following example.

A set of forty (40) glucose solutions each having a known concentration of glucose in water are prepared. The glucose concentration between each solution differs. Each glucose solution, in turn, is irradiated by each of the laser diodes 28. This creates a set of laser diode measurements for each glucose solution.

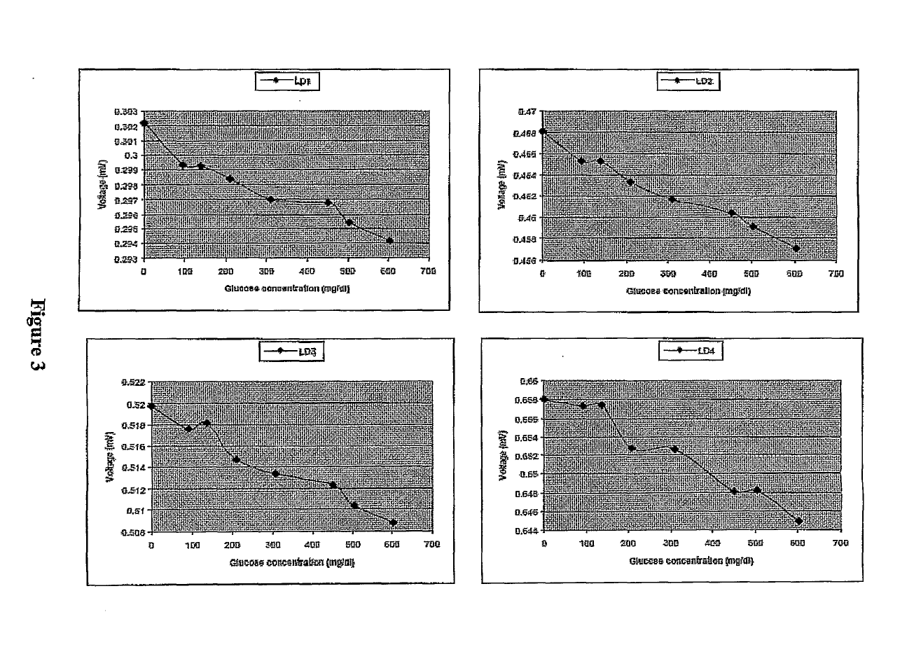

Once laser diode measurements have been taken for all the glucose solutions, the set of measurements taken by a laser diode for each glucose concentration is then plotted on a graph of glucose concentration versus laser diode voltage measurement. In the context of this example, representative graphs are produced and examples of such graphs for four laser diodes are shown in FIG. 3.

A manual review is then undertaken in respect of each graph and a “line of best fit” assessment made. The linear equation represented by the “line of best fit” is then calculated for each graph. The result is a set of six linear equations which are recorded with the data collection module 14 for use in training the first neural network 16.

In order to train the neural networks, a person 42 is requested to place his/her fingernail in the region delineated by the selector disc 24 and the detector disc 26. Once the fingernail is so placed, an operator (not shown) releases the clip from the selector disc 24. The operator then rotates the selector disc 24 until the aperture 34 is in co-axial alignment with the desired combination of laser diode 28 and fibre optic head 34. Once properly aligned, the laser diode 28 is activated so as to emit an infrared beam at the fingernail. The portion of the infrared beam not absorbed by glucose in the person's blood fluid is subsequently detected by the co-axially aligned fibre optic head 34. The fibre optic head 34 then provides a measurement reflective of the amount of infrared light received by it to the data collection module 14.

Once an infrared light measurement has been received by the data collection module 14 for the particular laser diode 28, the selector disc 24 is manipulated such that infrared light measurement for another laser diode 28 can be received. This process repeats until infrared light measurements have been received for each laser diode 28.

The whole process is repeated on the person at regular intervals a further fifty-nine times until a training set of sixty measurements are obtained. Each element of the training set comprises a set of six infrared light measurements. Each such infrared light measurement relates to a laser diode 28. To ensure that the training set does not include elements having substantially identical infrared light measurements, the person is required to consume a liquid that raises the blood glucose level over time prior to initiating the process that establishes the training set.

So as to set a benchmark blood glucose measurement for each element of the training set, at the same time that measurements are taken using the non-invasive blood glucose measurement setup 12, measurements are also taken using an invasive technique. In this embodiment, the invasive technique involves pricking the finger of the person and measuring the blood so obtained as would be known to a person skilled in the art. These sixty corresponding invasive blood glucose measurements form the verification set.

As mentioned above, the blood glucose measurements that form the training set and verification set are communicated to the data collection module 14. The data collection module 14 manipulates the data contained in both the training set and the verification set to form a training database 44. Each record 46 in the training database 44 comprises:
(i) An element from the training set. And
(ii) Its corresponding element in the verification set;

In this example, forty records 46 of the training database 44 are chosen at random and marked as training samples. The remaining twenty records are marked as testing samples.

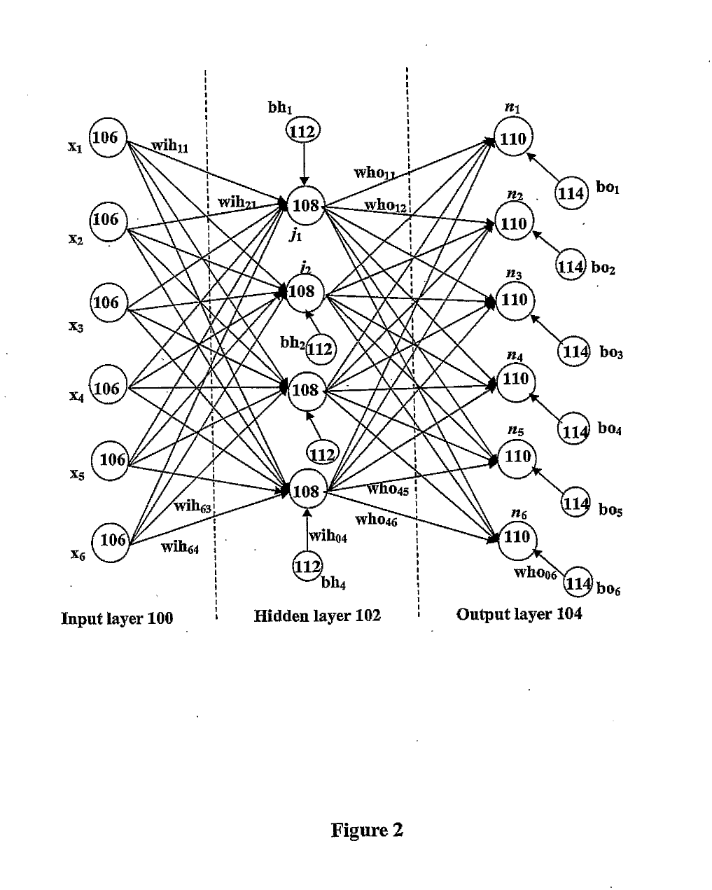

The records 46 marked as training samples are then used to train the first neural network 16. Training of the first neural network 16 will be described with reference to FIG. 2, where:
xᵢ represents the light measurement value representative of the i^{th} input node.
wihᵢⱼ represents the weight of the relationship between input node i and hidden node j. The weighting of the relationship between the bias node bhⱼ and each hidden node j is designated wih₀ⱼ.
bhⱼ represents the bias of hidden node j.
whoⱼₖ represents the weight of the relationship between hidden node j and k^{th} output node n. The weighting of the relationship between the bias node boₖ and each output node n is designated who₀ₖ.
boₖ represents the bias of the k^{th} output node n.
yᵢ represents the processed light measurement value representative of the i^{th} output node.

These notations remain consistent in the following training process for the first neural network which involves the following steps:
1. Each weight value (ie. wihᵢⱼ, and whoⱼₖ) is initialized. The initialization process involves assigning a random number in the range −0.5 to +0.5 to each weight value.
For this example, the weight values after initialization are as follows:

| | | | |
|---|---|---|---|
| wih_{₀₁} = −0.1954 | wih_{₂₁} = −0.2278 | wih_{₄₁} = 0.3462 | wih_{₆₁} = 0.3318 |
| wih_{₀₂} = −0.3103 | wih_{₂₂} = −0.3012 | wih_{₄₂} = 0.0252 | wih_{₆₂} = 0.0028 |
| wih_{₀₃} = −0.3066 | wih_{₂₃} = −0.4847 | wih_{₄₃} = −0.2974 | wih_{₆₃} = 0.2095 |
| wih_{₀₄} = 0.1822 | wih_{₂₄} = 0.2468 | wih_{₄₄} = 0.1721 | wih_{₆₄} = −0.0711 |
| wih_{₁₁} = −0.3611 | wih_{₃₁} = −0.0549 | wih_{₅₁} = 0.3381 | |
| wih_{₁₂} = −0.2972 | wih_{₃₂} = 0.4318 | wih_{₅₂} = −0.4804 | |
| wih_{₁₃} = −0.3013 | wih_{₃₃} = −0.0340 | wih_{₅₃} = 0.1813 | |
| wih_{₁₄} = 0.1038 | wih_{₃₄} = −0.0814 | wih_{₅₄} = −0.1205 | |

| | | |
|---|---|---|
| who_{₀₁} = 0.0466 | who_{₂₁} = 0.3537 | who_{₄₁} = 0.2271 |
| who_{₀₂} = −0.0551 | who_{₂₂} = 0.0936 | who_{₄₂} = −0.1907 |
| who_{₀₃} = 0.1946 | who_{₂₃} = −0.0034 | who_{₄₃} = 0.3385 |
| who_{₀₄} = 0.1213 | who_{₂₄} = 0.3998 | who_{₄₄} = 0.0681 |
| who_{₀₅} = 0.2948 | who_{₂₅} = 0.3216 | who_{₄₅} = −0.1296 |
| who_{₀₆} = −0.4568 | who_{₂₆} = 0.1449 | who_{₄₆} = 0.2027 |
| who_{₁₁} = 0.1972 | who_{₃₁} = 0.3180 | |
| who_{₁₂} = 0.0417 | who_{₃₂} = 0.1602 | |
| who_{₁₃} = −0.3491 | who_{₃₃} = −0.1580 | |
| who_{₁₄} = 0.1979 | who_{₃₄} = −0.2103 | |
| who_{₁₅} = −0.1216 | who_{₃₅} = −0.1588 | |
| who_{₁₆} = 0.3600 | who_{₃₆} = 0.0341 | |

2. Each bias value bhⱼ and boₖ are set to 1.
This example will now continue with reference to xᵢ values as follows:
   x₁=−0.8096 x₂=−0.2140 x₃=−0.7366
   x₄=−0.8120 x₅=−0.2866 x₆=−0.5204
These values have been obtained off a person having a blood glucose level of 5.95. Further, the equations for setting target values (tᵢ) for each xᵢ value are as follows:

t₁=−0.0129x₁+0.3996

t₂=−0.0130x₂+0.5072

t₃=−0.0380x₃+0.8920

t₄=−0.0159x₄+0.4271

t₅=−0.0079x₅+0.5377

t₆=−0.02642x₆+0.6863
Using these equations the target values tᵢ for each xᵢ value in this iteration of the first neural network is as follows:
   t₁=0.410 t₂=0.510 t₃=0.920
   t₄=0.440 t₅=0.540 t₆=0.700
3. The output value netⱼ for each hidden neuron j is calculated according to the following equation:

$${net}_{j} = {wih}_{0 j} \cdot {bh}_{j} + {\sum }_{i = 1}^{6} {wih}_{ij} {x}_{i}$$

In this example, the resulting netⱼ values are as follows:
   net₁=−0.3646 net₂=−0.2075 net₃=0.1466 net₄=0.0371
4. netⱼ is then normalized to obtain a f(netⱼ)) value. The f(netⱼ) value is attained in accordance with the following equation:

$$f ( {net}_{j} ) = \frac{1}{1 + exp ( - {net}_{j} )}$$

The f(netⱼ) values thus becomes a value in the range 0 to 1. In this example, the f(netⱼ) values are as follows:
   f(net₁)=0.4099 f(net₂)=0.4483
   f(net₃)=0.5366 f(net₄)=0.5093
5. The output value netoₖ for output neuron nₖ is then computed according to the following equation:

$${neto}_{k} = {who}_{{0}^{k}} \cdot {bo}_{k} + {\sum }_{j = 1}^{4} {who}_{jk} \cdot f ( {net}_{j} )$$

This produces the following netoₖ values:
   neto₁=0.4106 neto₂=−0.0072 neto₃=0.1376
   neto₄=0.3035 neto₅=0.2379 neto₆=−0.1288
6. The value of netoₖ is thereafter normalized to obtain a nₖ value. Nₖ is computed to a value between 0 and 1 according to the following equation:

$${n}_{k} = \frac{1}{1 + exp ( - {neto}_{k} )}$$

The resulting values are thus:
   n₁=0.6012 n₂=0.4982 n₃=0.5343
   n₄=0.5753 n₅=0.5592 n₆=0.4693
7. Once the neural network output nₖ is obtained, the output gradient error δₖ for the k^{th} output neuron in output layer is computed according to the following equation:

δₖ=(tₖ−nₖ)·nₖ·(1−nₖ)
This results in the following output gradient error (δₖ) values:
   δ₁=−0.0458 δ₂=0.0030 δ₃=0.0960
   δ₄=−0.0331 δ₅=−0.0047 δ₆=0.0574
8. The output gradient error δₖ is also required to compute the hidden layer gradient error δⱼ to be used in the current iteration of the first neural network. This is calculated by the following equation:

$${δ}_{j} = ( f ( {net}_{j} ) \cdot ( 1 - f ( {net}_{j} ) ) ) {\sum }_{k = 1}^{6} {δ}_{k} \cdot {who}_{jk} ( p )$$

where:
   whoⱼₖ(p) represents the whoⱼₖ value used in the current iteration of the first neural network.
This produces the following set of values:
   δ₁=−0.0023
   δ₂=−0.0056
   δ₃=−0.0049
   δ₄=0.0079
9. The output gradient error δₖ is required to compute the change in weight Δwhoⱼₖ to be used in the next iteration of the first neural network. This change is calculated by the following equation:

Δwhoⱼₖ(p+1)=η·δₖ·f(netⱼ)+m·Δwhoⱼₖ(p)
where:
   η denotes the learning rate.
   m denotes the momentum composition.
   Δwhoⱼₖ(p+1) represents the updated change in weight.
   Δwhoⱼₖ(p) represents the previous change in weight.
This produces the following set of values:

| | | | |
|---|---|---|---|
| Δwho_{₁₁} = −0.0019 | Δwho_{₂₁} = −0.0021 | Δwho_{₃₁} = −0.0025 | Δwho_{₄₁} = −0.0023 |
| Δwho_{₁₂} = 0.0001 | Δwho_{₂₂} = 0.0001 | Δwho_{₃₂} = 0.0002 | Δwho_{₄₂} = 0.0002 |
| Δwho_{₁₃} = 0.0039 | Δwho_{₂₃} = 0.0043 | Δwho_{₃₃} = 0.0051 | Δwho_{₄₃} = 0.0049 |
| Δwho_{₁}_{₄} = −0.0014 | Δwho_{₂₄} = −0.0015 | Δwho_{₃₄} = −0.0018 | Δwho_{₄₄} = −0.0017 |
| Δwho_{₁₅} = −0.0002 | Δwho_{₂₅} = −0.0002 | Δwho_{₃₅} = −0.0003 | Δwho_{₄₅} = −0.0002 |
| Δwho_{₁}_{₆} = 0.0024 | Δwho_{₂₆} = 0.0026 | Δwho_{₃₆} = 0.0031 | Δwho_{₄₆} = 0.0029 |

It is noted that this formula is a recursive function. In order to facilitate this recursive function, each whoⱼₖ value is stored in an array for reference by future iterations of the first neural network.
The terms η and m, will be used throughout the remainder of this specification to denote the learning rate and momentum composition, respectively.
10. The weightings of the output bias values who₀ₖ are then revised by first determining the correction values according to the following formula:

Δwho₀ₖ=η·δₖ
The resulting correction values are:
   Δwho₀₁=−0.0046 Δwho₀₂=0.0003 Δwho₀₃=0.0096
   Δwho₀₄=−0.0033 Δwho₀₅=−0.0005 Δwho₀₆=0.0057
11. Having calculated δⱼ it is then possible to calculate the correction values for wihᵢⱼ using the following formula:

Δwihᵢⱼ(p+1)=η·δⱼxᵢ+mΔwihᵢⱼ(p)

| | | | |
|---|---|---|---|
| Δwih_{₁₁} = −0.0002 | Δwih_{₂₃} = 0.0005 | Δwih_{₄₁} = 0.0004 | Δwih_{₅₃} = −0.0006 |
| Δwih_{₁₂} = 0.0000 | Δwih_{₂₄} = 0.0001 | Δwih_{₄₂} = 0.0001 | Δwih_{₅₄} = −0.0002 |
| Δwih_{₁₃} = 0.0002 | Δwih_{₃₁} = 0.0004 | Δwih_{₄₃} = 0.0004 | Δwih_{₆₁} = −0.0006 |
| Δwih_{₁₄} = 0.0002 | Δwih_{₃₂} = 0.0005 | Δwih_{₄₄} = 0.0004 | Δwih_{₆₂} = −0.0006 |
| Δwih_{₂₁} = 0.0001 | Δwih_{₃₃} = 0.0002 | Δwih_{₅₁} = 0.0001 | Δwih_{₆₃} = −0.0002 |
| Δwih_{₂₂} = 0.0001 | Δwih_{₃₄} = 0.0003 | Δwih_{₅₂} = 0.0003 | Δwih_{₆₄} = −0.0004 |

As this is also a recursive function, each wihᵢⱼ value is stored in an array for reference by future iterations of the first neural network 16.
12. The bias weighting correction values wih₀ⱼ are then determined using the following formula:

Δwih₀ⱼ=η·δⱼ
The resulting correction values are:
   Δwih₀₁=−0.0002 Δwih₀₂=−0.0006
   Δwih₀₃=−0.0005 Δwih₀₄=0.0008
13. With the correction values determined, the weightings whoⱼₖ are updated according to the following formula:

whoⱼₖ(p+1)=whoⱼₖ(p)+Δwhoⱼₖ(p+1)
This equation also applies to the bias weighting values, thus resulting in a new set of weightings as follows:

| | | |
|---|---|---|
| who_{₀₁} = 0.0420 | who_{₂₁} = 0.3516 | who_{₄₁} = 0.2248 |
| who_{₀₂} = −0.0548 | who_{₂₂} = 0.0937 | who_{₄₂} = −0.1905 |
| who_{₀₃} = 0.2042 | who_{₂₃} = 0.0009 | who_{₄₃} = 0.3434 |
| who_{₀₄} = 0.1180 | who_{₂₄} = 0.3983 | who_{₄₄} = 0.0664 |
| who_{₀₅} = 0.2943 | who_{₂₅} = 0.3214 | who_{₄₅} = −0.1298 |
| who_{₀₆} = −0.4511 | who_{₂₆} = 0.1475 | who_{₄₆} = 0.2056 |
| who_{₁₁} = −0.1991 | who_{₃₁} = 0.3155 | |
| who_{₁₂} = 0.0418 | who_{₃₂} = 0.1604 | |
| who_{₁₃} = −0.3452 | who_{₃₃} = −0.1529 | |
| who_{₁₄} = 0.1965 | who_{₃₄} = −0.2121 | |
| who_{₁₅} = −0.1218 | who_{₃₅} = −0.1591 | |
| who_{₁₆} = 0.3624 | who_{₃₆} = 0.0372 | |

14. In an almost identical manner, the weightings wihᵤ are updated according to the following formula:

wihᵢⱼ(p+1)=wihᵢⱼ(p)+Δwihᵢⱼ(p)+Δwihᵢⱼ(p+1)
This equation also applies to the bias weighting values, thus resulting in a new set of weightings as follows:

| | | | |
|---|---|---|---|
| wih_{₀₁} = −0.1956 | wih_{₂₁} = −0.2278 | wih_{₄₁} = 0.3464 | wih_{₆₁} = 0.3319 |
| wih_{₀₂} = −0.3109 | wih_{₂₂} = −0.3011 | wih_{₄₂} = 0.0257 | wih_{₆₂} = 0.0031 |
| wih_{₀₃} = −0.3071 | wih_{₂₃} = −0.4846 | wih_{₄₃} = −0.2970 | wih_{₆₃} = 0.2098 |
| wih_{₀₄} = 0.1830 | wih_{₂₄} = 0.2466 | wih_{₄₄} = 0.1715 | wih_{₆₄} = −0.0715 |
| wih_{₁₁} = −0.3609 | wih_{₃₁} = −0.0547 | wih_{₅₁} = 0.3382 | |
| wih_{₁₂} = −0.2967 | wih_{₃₂} = 0.4322 | wih_{₅₂} = −0.4802 | |
| wih_{₁₃} = −0.3009 | wih_{₃₃} = −0.0336 | wih_{₅₃} = 0.1814 | |
| wih_{₁₄} = 0.1032 | wih_{₃₄} = −0.0820 | wih_{₅₄} = −0.1207 | |

Processing then commences again at step 3 with a new set of xᵢ values taken from the training set.

This process continues with xᵢ values taken from the training set being used or re-used as needed until such time as the global mean square error of the system is less than 0.0008. Typically, this is attained after several thousands of iterations.

Once the first neural network has been trained, the second neural network is trained in an identical fashion, with the exception that there is only one output node n₁. As such, a description of the processing needed to train the second neural network will not be repeated here. Once trained, the output layer values calculated by the first neural network are used as the xᵢ values for the second neural network.

Once both neural networks have been trained using the training sets, the system as a whole is tested using the values contained in the verification set. If the system as tested using the verification set shows significant error, then the system is retrained using a new training set more representative of the verification set.

A second embodiment of the system 10 for analysing measurements of a composition of a blood fluid, where like numerals reference like parts, will now be described. The system 10 comprises a data collection module 14, a first neural network 16 and a second neural network 18. The invention will now be described in the context of analysing measurements of blood glucose level in the blood fluid with the objective of determining an overall measurement of the composition in the blood fluid. Additional features necessary to the operation of the system 10 may also be introduced in the context of the following example.

The data collection module 14 is configured to receive the following information.

a. A set of sixty non-invasive blood glucose measurements obtainable via any non-invasive blood glucose measurements means. The set of sixty non-invasive blood glucose measurements forms the training set.

b. A set of linear equations. Each linear equation depicts the relationship between varying level of blood glucose solutions and the unit of measurement of the non-invasive blood glucose measurements means. In the context of this embodiment, the non-invasive blood glucose measurements means is the blood measurement setup 12 as described in the first embodiment, thus six linear equations corresponding to the six laser diodes are obtained.

c. A corresponding benchmark blood glucose measurement for each element of the training set, that measurements are taken using an invasive technique such as that which involves pricking the finger of the person and measuring the blood so obtained as would be known to a person skilled in the art. These sixty corresponding invasive blood glucose measurements form the verification set.

The data collection module 14 manipulates the data contained in both the training set and the verification set to form a training database 44. Each record 46 in the training database 44 comprises:
(iii) An element from the training set. And
(iv) Its corresponding element in the verification set;

In this example, forty records 46 of the training database 44 are chosen at random and marked as training samples. The remaining twenty records are marked as testing samples.

The records 46 marked as training samples are then used to train the first neural network 16. Training of the first neural network 16 will be described with reference to FIG. 2, where:
xᵢ represents the light measurement value representative of the 1^{th} input node.
wihᵢⱼ represents the weight of the relationship between input node i and hidden node j. The weighting of the relationship between the bias node bhⱼ and each hidden node j is designated wih₀ⱼ.
bhⱼ represents the bias of hidden node j.
whoⱼₖ represents the weight of the relationship between hidden node j and k^{th} output node n. The weighting of the relationship between the bias node boₖ and each output node n is designated who₀ₖ.
boₖ represents the bias of the k^{th} output node n.
yᵢ represents the processed light measurement value representative of the i^{th} output node.

These notations remain consistent in the following training process for the first neural network which involves the steps 1 to 14 as described in the first embodiment. The training process then commences again at step 3 with a new set of xᵢ values taken from the training set.

This process iterates and continues with xᵢ values taken from the training set being used or re-used as needed until such time as the global mean square error of the system is less than 0.0008. Typically, this is attained after several thousands of iterations.

Once the first neural network has been trained, the second neural network is trained in an identical fashion, with the exception that there is only one output node n₁. As such, a description of the processing needed to train the second neural network will not be repeated here. Once trained, the output layer values calculated by the first neural network are used as the xᵢ values for the second neural network.

Once both neural networks have been trained using the training sets, the system as a whole is tested using the values contained in the verification set. If the system as tested using the verification set shows significant error, then the system is retrained using a new training set more representative of the verification set. The trained neural networks verified by the verification set provide an overall measurement of the composition of blood glucose representative of the blood glucose level in the blood fluid.

It should be appreciated by the person skilled in the art that the invention is not limited to the examples described. In particular, the following additions and/or modifications can be made without departing from the scope of the invention:
At least one control laser diode(s) may be added to the wavelength source disc 22. The control laser diodes(s) may also replace either one of the six laser diodes 28. The control laser diode(s) is configured to emit an infrared wavelength that is not absorbable by glucose. Based on current knowledge, such wavelengths that fall within the range 1600 nm to 2200 nm as absorbable by glucose.
The control laser diode(s) may be used to determine the base intensity of infrared wavelength measured when no glucose are absorbed. Correspondingly, a control electrical voltage reading may be obtained and processed using signal processor 48.
The rotation of the wavelength selector disc 24 may be performed manually, or may be automated using for example, a stepper motor.
Instead of using six laser diodes 28, with each laser diode 28a, 28b, 28c, 28d, 28e, 28f emitting a fixed infrared wavelength, a single laser diode capable of emitting a plurality of varying infrared wavelengths may be used.
Either more or less laser diode(s) may be added or removed from the wavelength source disc.
Instead of the fingernail bed, the region of diagnosis may be any part of the person 42 known to be suitable for diagnosis by a person skilled in the art.
The system 10 may be used for the measurement of other compositions in the blood fluid besides glucose. In such alternative setup, the infrared wavelengths emitted by six laser diodes 28 is required to be re-calibrated and optimized to the composition's peak absorption wavelength.
The non-invasive blood glucose measurement setup 12 may be replaced by any alternative configuration for non-invasive blood glucose measurement as is known to a person skilled in the art.
The stopping criteria for stopping the training process of the neural networks 16, 18 may be any which is known to the person skilled in the art. Some examples include the consideration of absolute rate of change in mean squared error per training set; stability of synaptic weights and bias level; mean squared error over the entire training set, fixed number of iterations, etc.
The learning rate η and momentum constant m for each epoch p may be determined based on any set of rules known and obvious to the skilled person.
Alternative activation function(s) well known by a skilled person may be adopted in replacement of the sigmoidal activation function. However, these activation functions should be differentiable.
While the learning rate and momentum compositions can be any value between 0 and 1, more accurate results have been achieved where there is some trade off between the learning rate and momentum composition. The best results have been achieved where the learning rate is a value between 0.01 and 0.1, while the momentum composition is within the range 0.8 to 0.9.
The learning rate and momentum composition may be manually adjusted at any stage during training of either the first or second neural network. Typically, the learning rate is adjusted in situations where the error is oscillating.
To ensure the greatest accuracy in training of the neural networks, the training set should provide representative samples from varying ranges of blood glucose measurements. In order to do this, some manual intervention may be required.
The number of nodes in the hidden layer included in either neural network may be any number in excess of four.
The number of decimal places used for determining the weightings of each link in the neural networks may vary. However, for accuracy reasons, it has been determined that a minimum of three decimal places should be used.
The bias and bias weightings can be eliminated. However, it is believed that doing so may mean that the time needed to train a neural network will be increased.
The weightings may fall within other range sets beyond the −0.5 to 0.5 mentioned above. For instance, a weight value range of −0.25 to 0.25 may also be used.
While the invention as described in this specification has been illustrated with reference to one form of a back propagation algorithm, it should be appreciated that the invention is not limited to the use of this particular variant. Other variant back propagation algorithms may be used and such fall within the scope of the present invention.
It is also possible to use other activation functions to those described above without departing from the scope of the present invention. It is understood that any activation function that limits the resulting values to the range −1 to 1 may be used.
Training of the systems described above are examples of a sequential training mode. However, it is equally as possible to undertake training in batch mode. In such a situation, weightings are adjusted after the entire training set has been presented to the neural network being trained.
In a further variation of the above embodiment, the glucose solutions may be omitted. In its place a linear equation set is established out of the training set of blood glucose measurements. Ideally, this linear equation set has forty elements. The linear equations are then determined manually by plotting a graph for each laser diode of the signal voltage reading against the known blood glucose level (as determined by the invasive blood glucose measurement system). A “line of best” fit is then determined from the plotted graph.

It should be further appreciated by the person skilled in the art that features and modifications discussed above, not being alternatives or substitutes, can be combined to form yet other embodiments that fall within the scope of the invention described.

1. A system for measuring a composition of a blood fluid comprising:
at least one neural-network for processing a plurality of measurements taken by a non-invasive measuring unit to determine an overall measurement of the composition in the blood fluid, wherein a linear equation associated with each output node of the at least one neural network is determined from a controlled source prior to training the at least one neural network.

2. A system for measuring a composition of a blood fluid comprising:
a non-invasive measuring unit for measuring the composition; and
at least one neural network for processing a plurality of measurements taken by the non-invasive measuring unit to determine an overall measurement of the composition in the blood fluid, wherein a linear equation associated with each output node of the at least one neural network is determined from a controlled source prior to training the at least one neural network.

3. A method of measuring a composition in a blood fluid comprising:
Obtaining a plurality of measurements from a non-invasive measuring unit, and
processing the plurality of measurements by at least one neural network to determine an overall measurement of the composition in the blood fluid, wherein a linear equation associated with each output node of the at least one neural network is determined from a controlled source prior to training the at least one neural network.

4. A system or method for measuring a composition in the blood fluid according to any of claim 1 to 3, wherein the linear equation associated with each output node is obtained based on a line of best fit.

5. A system or method for measuring a composition in the blood fluid according to any of claim 1 to 4, where the linear equation associated with each hidden node is determined through automated processes.

6. A system or method for measuring a composition in the blood fluid according to any one of claims 1 to 5, where the at least one neural network implements a back propagation algorithm.

7. A system or method for measuring a composition in the blood fluid according to claim 5, where the number of nodes in an input layer of the at least one neural network matches the number of measurements in the plurality of measurements taken by the non-invasive measuring unit.

8. A system or method for measuring a composition in the blood fluid according to any one of claim 5 to 7, where the at least one neural network comprises at a hidden layer of at least four nodes.

9. A system or method for measuring a composition in the blood fluid according to any one of claims 6 to 8, where the output value for the hidden node is a summation of weighted measurements.

10. A system or method for measuring a composition in the blood fluid according to any one of claims 6 to 9, where the output value for the output node is a summation of weighted normalized hidden node output values.

11. A system or method for measuring a composition in the blood fluid according to any one of claims 6 to 10, where the adjustments to the weightings for each link between a hidden node and an output node are calculated with reference to an output gradient error.

12. A system or method for measuring a composition in the blood fluid according to claim 11, where the output gradient error is calculated as follows:
δₖ=(tₖ−nₖ)·nₖ·(1−nₖ)
where:
δₖ is the output gradient error
nₖ is the normalized output value for output node k.
tₖ is the target output value for output node k as determined by the linear equation associated with output node k.

13. A system or method for measuring a composition in the blood fluid according to claim 12, where the adjustment to the weightings for each link between a hidden node and an output node are calculated according to the formula:
Δwhoⱼₖ(p+1)=η·δₖ·f(netⱼ)+m·Δwhoⱼₖ(p)
where:
η denotes the learning rate.
m denotes the momentum composition.
δₖ is the output gradient error.
whoⱼₖ(p+1) represents the updated change in weight.
whoⱼₖ(p) represents the previous change in weight.
f(netⱼ) is the normalized output value for hidden node j.

14. A system or method for measuring a composition in the blood fluid according to any one of claims 6 to 13, where the adjustments to the weightings for each link between an input node and a hidden node are calculated with reference to a hidden layer gradient error.

15. A system or method for measuring a composition in the blood fluid according to claim 14, where the hidden layer gradient error is calculated as follows:
$${δ}_{j} = ( f ( {net}_{j} ) \cdot ( 1 - f ( {net}_{j} ) ) ) {\sum }_{k = 1}^{Y} {δ}_{k} \cdot {who}_{jk} ( p )$$
where:
Y is the total number of neurons in the output layer of the neural network concerned.
f(netⱼ) is the normalized output value for hidden node j.
δₖ is the output gradient error.
whoⱼₖ(p) represents the current weight for the link between the hidden node j and the output node k.

16. A system or method for measuring a composition in the blood fluid according to claim 15, where the adjustments to the weightings for each link between an input node and a hidden node are calculated as follows:
Δwihᵢⱼ(p+1)=η·δⱼ·xᵢ+mΔwihᵢⱼ(p)
where:
η denotes the learning rate.
m denotes the momentum composition.
δⱼ is the hidden layer gradient error.
xᵢ is the value of input node i.
wihᵢⱼ(p+1) represents the updated change in weight.
wihᵢⱼ(p) represents the previous change in weight.

17. A system or method for measuring a composition in the blood fluid according to claim 13 or claim 16, where the learning rate (η) and the momentum parameter (m) are automatically adjusted during training.

18. A system or method for measuring a composition in the blood fluid according to claim 13, claim 16 or claim 17, where the learning rate (η) is a value in the range 0.01 to 0.1 and the momentum parameter (m) is a value in the range 0.8 to 0.9.

19. A system or method for measuring a composition in the blood fluid according to any one of claims 6 to 18, where the at least one neural network comprises at least one bias.

20. A system or method for measuring a composition in the blood fluid according to claim 19, as dependent on claim 9, where the output value for the hidden node is a summation of weighted measurements and at least one weighted input bias.

21. A system or method for measuring a composition in the blood fluid according to claim 19, as dependent on claim 10, where the output value for the output node is a summation of weighted normalized hidden node output values and at least one weighted output bias.

22. A system or method for measuring a composition in the blood fluid according to claim 19, as dependent on claim 11, where the adjustments to the weightings of each link between each output bias and an output node is calculated with reference to the output gradient error.

23. A system or method for measuring a composition in the blood fluid according to claim 19, as dependent on claim 12, where each link weighting is calculated according to the following formula:
Δwho₀ₖ₌η·δₖ
where:
η is the learning rate.
δₖ is the output gradient error.

24. A system or method for measuring a composition in the blood fluid according to claim 23, where the adjustment to be made to the output value for the output node (netoₖ) is determined by the following equation:
$${neto}_{k} = {who}_{{0}^{k}} \cdot {bo}_{k} + {\sum }_{j = 1}^{4} {who}_{jk} \cdot f ( {net}_{j} )$$
where:
X is the total number of nodes in the hidden layer of the neural network concerned
who₀ₖ is the weighting applied to the output bias for output node k.
boₖ is the output bias for output node k.
whoⱼₖis the weighting applied to the link between hidden node j and output node k.
f(netⱼ) is the normalized output value for hidden node j.

25. A system or method for measuring a composition in the blood fluid according to any preceding claim, where the at least one neural network comprises a first neural network and a second neural network, the first neural network configured so as to pre-process the plurality of measurements before passing the pre-processed measurements to the second neural network for determination of an overall measurement of the composition.

26. A system or method for measuring a composition in the blood fluid according to claim 25, where the first and second neural networks implement back propagation algorithms.

27. A system or method for measuring a composition in the blood fluid according to claim 26 where the first and second neural networks implement the same back propagation algorithm.

28. A system or method for measuring a composition in the blood fluid according to any one of claims 6 to 27, where the at least one neural network is trained until one of the following occurs: the mean square error per training set is within a predetermined range; the synaptic weights stabilise; the bias level stabilises; the mean square error of the system is within a predetermined range; the mean square error over the entire training set is within a predetermined range; a predetermined number of training iterations have been performed.

29. A system or method for measuring a composition in the blood fluid according to claim 28, where the at least one neural network is trained until the global mean square error of the system is less than 0.0008.

30. A system or method for measuring a composition in the blood fluid according to any preceding claim, where after training of the at least one neural network, the neural networks are verified by comparing the results of the trained neural network against measurements of the substance obtained through invasive measuring techniques.

31. A system or method for measuring a composition in the blood fluid according to any of claim 2-30 , where the non-invasive measuring unit comprises a plurality of laser diodes each emitting light at a unique wavelength absorbable by the composition, the measurements taken by each laser diode forming the plurality of measurements.

32. A system or method for measuring a composition in the blood fluid according to claim 31, where the composition to be measured is blood glucose and the wavelength of the light emitted by each of the plurality of laser diodes falls within the range 1600 nm to 1800 nm.

33. A system or method for measuring a composition in the blood fluid according to any one of claims 2 to 30, where the non-invasive measuring unit comprises at least one laser diode able to emit light at varying wavelengths absorbable by the composition, the measurements taken by the at least one laser diode at each of these varying wavelengths forming the plurality of measurements.

34. A system or method for measuring a composition in the blood fluid according to any one of claims 30 to 32, where the non-invasive measuring unit further comprises a control laser diode which emits light at a wavelength not absorbable by the composition.

35. A computer-readable medium having recorded thereon:
Means for receiving a plurality of measurements of a composition of a blood fluid, and
at least one neural network to process the plurality of measurements of the composition of the blood fluid,
such that an overall measurement of the composition in the blood fluid is determined, wherein a linear equation associated with each output node of the at least one neural network is determined from a controlled source prior to training the at least one neutral network.
